(19) United States
(12) Patent Application Publication
(10) Pub. No.: US 2017/0102337 A1
(43) Pub. Date: Apr. 13, 2017

(54) SELF-FUSING SILICONE TAPE FOR SENSING HYDROGEN AND HYDROGEN SULFIDE GAS LEAKS

(71) Applicant: ELEMENT ONE, INC., Boulder, CO (US)

(72) Inventors: David K. Benson, Golden, CO (US); Mark C. Hatje, West Haven, CT (US); William Hoagland, Boulder, CO (US); John Poplawski, Meriden, CT (US)

(21) Appl. No.: 15/346,615

(22) Filed: Nov. 8, 2016

Related U.S. Application Data
(63) Continuation-in-part of application No. 14/597,294, filed on Jan. 15, 2015.
Application No. 14/597,294 is a continuation-in-part of application No. 13/273,405, filed on Oct. 14, 2011, now abandoned.
Application No. 13/273,405 is a continuation-in-part of application No. 13/166,777, filed on Jun. 22, 2011, now Pat. No. 9,422,160.
Application No. 13/166,777 is a continuation-in-part of application No. 11/553,400, filed on Oct. 26, 2006, now abandoned.
(60) Provisional application No. 62/254,562, filed on Nov. 12, 2015.

(51) Int. Cl. G01N 21/78; G01N 33/00

(52) U.S. Cl. CPC G01N 21/783; G01N 33/005; G01N 33/0044; G01N 2021/7759

(57) ABSTRACT

This invention relates to a tape product, and method of manufacturing the product, which incorporates a gas sensing pigment that causes the tape to change color upon exposure to gases such as hydrogen or hydrogen sulfide. The gas sensing pigment is compounded together with a silicone rubber to produce a self-fusing silicone tape which may be applied in a variety of industrial environments where hydrogen or hydrogen sulfide gas leakage is a concern. The tape may be colored to enhance visibility and/or to indicate which hazardous gas it is intended to detect. The tape may be fiber reinforced with carbon fiber to increase its longitudinal strength for industrial applications. Periodic perforations across the width of the tape may be added to facilitate the tearing or cutting of convenient lengths of the tape.

### CROSS-REFERENCE TO RELATED APPLICATIONS

This is a continuation-in-part claiming the priority benefit under 35 U.S.C. 119(e) of U.S. Provisional Application No. 62/254,562 filed Nov. 12, 2015, and under 35 U.S.C. 120 of U.S. patent application Ser. No. 14/597, 294, Self-Fusing Carbon Fiber Silicone Perforated Tape, filed Jan. 15, 2015, which is a continuation-in-part of U.S. patent application Ser. No. 13/273,405 entitled Self-Fusing Carbon Fiber Silicone Tape and Manufacturing Process, filed Oct. 7, 2011 (abandoned), and U.S. Pat. No. 9,422,160, entitled Visual Hydrogen Sensors Using Chemochromic Metal Oxide Microparticles Superficially Coated With Catalyst, filed Jun. 22, 2011, U.S. patent application Ser. No. 11/553,400 filed Oct. 26, 2006 entitled “Visual Hydrogen Sensors Using Nanoparticles,” which claims the benefit of U.S. Provisional Patent Application Ser. No. 60/730,960 filed Oct. 28, 2005 entitled “Hydrogen Indicating Pigments to Detect Hydrogen Gas,” all of the entire disclosures of which are hereby incorporated by reference.

### FIELD OF THE INVENTION

The present invention relates generally to self-sealing silicone tape that changes color in the presence of toxic, flammable and explosive gases such as hydrogen and hydrogen sulfide.

### BACKGROUND

Self-sealing silicone tape products are generally used to secure pipe joints to prevent or stop liquid leaks. The present invention discloses improvements upon the well-known silicone self-sealing tape to extend its use to the detection of toxic and potentially explosive gases such as hydrogen and hydrogen sulfide that might leak from pressurized gas pipes and fittings.

Hydrogen gas is used widely as a feed chemical in the chemical processing industry, petroleum processing industry, metal refining and processing, semiconductor device fabrication, food processing, and more recently as a fuel for vehicles. World-wide use of hydrogen gas is estimated to be greater than 300 billion cubic meters annually. Hydrogen sulfide is more commonly an impurity or waste product in various industrial processes such as metal refining, municipal waste processing, paper making, and in natural gas recovery and processing. Hydrogen sulfide impurities in natural gas is typically large enough that it may be used as a tracer gas for locating natural gas leaks in gathering fields and processing plants. Both hydrogen and hydrogen sulfide gases are highly flammable and form explosive mixtures with air. Hydrogen sulfide is particularly toxic, causing rapid death if breathed in high concentrations.

Because of their hazardous nature, hydrogen or hydrogen sulfide gas leaks must be detected and repaired promptly to prevent serious accidents. Typically such leaks are detected by electronic gas sensors that are connected to a central safety alarm system that triggers automatic responses such as closing valves, shutting down processes, sounding alarms, turning on ventilation fans, etc. Such leak detectors are expensive and difficult to connect to central controls, particularly in an expansive processing facility. For this reason, the electronic leak detectors are sparsely deployed and each one may monitor a large space with multiple potential leak sites. Only if a substantial leak develops in the monitored space is the concentration of the leaking gas likely to reach a high enough level to trigger an alarm. By that time, the situation is a true safety issue and the entire process is commonly shut down until the exact source of the leak can be found and repaired. This is commonly a very costly event in terms of lost production as well as the labor cost to locate and repair the leak.

### SUMMARY OF THE INVENTION

The present invention discloses a modified self-sealing silicone hydrogen indicating tape that can be economically used as a complement to conventional electronic gas leak detectors. Unlike the electronic hazardous gas detectors, an indicator tape requires no wiring or no electric power. It is also economical to produce and use. A silicone tape hydrogen or hydrogen sulfide gas indicator may be used as a complement to the conventional electronic hydrogen or hydrogen sulfide gas detectors and thereby improving the safety of a facility.

The tape contains a pigment, which reacts with a hazardous gas by changing color. By applying the self-sealing tape to potential gas leak sites such as pipe joints, valve stems, pipe flanges, etc., a process operator can gain an early warning of a gas leak before they become large enough to trigger an electronic alarm (with its attendant process shutdown, etc.). If a leak begins to develop in a pipe fitting, the tape around that fitting will change color and provide an indication during routine inspections that the fitting needs repair or replacement. A repair as simple as tightening the bolts on a flange may be sufficient. If more extensive repairs are needed, they can be planned and scheduled to minimize shutdown time and loss of productivity, as opposed to an abrupt, unplanned emergency shutdown if the leak had been allowed to grow large enough to trigger the electronic gas leak detector.

Self-sealing silicone tapes have several advantages over more conventional non-self sealing adhesive tapes. Silicone compounds are highly permeable to hydrogen gas allowing chemochromic pigments to be mixed into the compound, rather than applied as a coating, where it is protected from environmental factors that negatively affect it such as ultraviolet (UV) sunlight, water, contaminants, etc., while remaining accessible to molecular hydrogen or other gases of interest via permeation through the silicone rubber. Conventional tape products utilizing adhesives to secure tape to a pipe or to an underlying layer of tape have proven unreliable in many industrial uses, especially where the tape is exposed to severe weather or where chemicals, such as oils and solvents might be present and deteriorate the adhesive compounds. It has been found that a mechanically pliable self-fusing silicone rubber tape, one that stretches to conform to the pipe or fitting and securely bonds to itself, is more reliable than adhesive tapes.

Where the silicone tape overlaps itself, it bonds by polymerizing with the underlying tape layer. Once the polymerization is complete (typically within about 24 hours), the silicone tape wrap is a single coherent encapsulating polymer wrap. The silicone polymer is highly resistant to water, industrial chemicals and severe weather conditions and remains durable at operating temperatures up to 300° C.

The leak indicating tape is extremely durable and may be left in place under typical outdoor weather conditions for extended periods of one year or even longer. For higher temperatures above 200° C. up to 260° C., the invention will reliably indicate the presence of a hazardous gas leak of interest (e.g. hydrogen) for shorter periods of from several months to at least 24 hours, making it useful for spot testing on high temperature piping, valves and fittings.

The present invention discloses improvements in self-sealing silicone tape which make it useful for detecting leaks of hazardous gases such as hydrogen and hydrogen sulfide. The improved tape incorporates chemically reactive pigments in the silicone which react with the hazardous gas and change color. In applications, this distinctive change in color provides a clearly visible early warning of a gas leak.

Organic rubber has a carbon to carbon backbone which leaves them susceptible to ozone, UV radiation, heat and other ageing factors that silicone rubber withstands well. At extreme temperatures, the tensile strength, elongation, tear strength and compression set can be far superior to conventional rubbers although still low relative to other materials. However, compared to organic rubbers, silicone rubber tape has a very low tensile strength. For tape products that need to withstand even relatively low imposed loads, there is a need for a self-fusing tape that has a relatively high tensile strength. As used herein, the term “tensile strength” is a quantitative measure of the pulling force a certain length of tape can exert without breaking.” This definition of tensile strength must be contrasted from “modulus of elasticity.” The term “modulus of elasticity” as used herein means a measure of a tape's resistance to stretching, the higher the number the stiffer its resistance to stretching. In many silicone tape applications, the lack of a tape's resistance to stretching is preferred. Therefore, any improvement in a silicone tape, should strive to maintain the tape's maximally achievable modulus of elasticity and wherever possible improve the elastic limit, or yield strength, where a permanent deformation in a silicone tape will occur. Silicone tapes that resist tearing, or have high tear strength, are also desirable in certain applications, and therefore any improvement in a silicone tape, such as the incorporation of fibers, serve to maintain the tape's maximally achievable tear resistance.

Carbon fiber may be employed to improve tear resistance in industrial applications. Carbon fiber tows are comprised of thin strands measuring between 0.005-0.010 mm in diameter, composed mostly of carbon atoms. The carbon atoms are bonded together in microscopic crystals that are more or less aligned parallel to the long axis of the fiber. The crystal alignment makes the fiber very strong for its size.

Notably several thousand carbon fibers twisted together form a tow, thread or yarn, which is, as more fully described below, used in the invention herein to create a tape product that improves the tensile strength by embedding the carbon fibers between layer of self-fusing silicone tape, while keeping the “modulus of elasticity” unchanged from the silicone tape products that do not contain the feature of having embedded carbon fibers.

Additional coloration may be added to the tape as a whole or as a marking along the length of the tape to provide additional information to the user and/or to increase its visibility in the field. The tape may be color coded with additional pigment or dye to indicate which hazardous gas it is intended to detect. Fluorescent pigment or dye may be added to the tape to enhance its visibility.

### BRIEF DESCRIPTION OF THE DRAWINGS

Understanding of the present invention will be facilitated by consideration of the following detailed description of the preferred embodiments of the invention taken in conjunction with the accompanying illustrations, in which like numerals refer to like parts, and wherein:

FIG. 1A, FIG. 1B show images of a portion of a self-fusing tape product incorporating a hydrogen gas sensor and a catalyst, in accordance with an embodiment of the invention;

FIG. 2A and FIG. 2B show images of the self-fusing gas leak indicating tape product applied to a piping fitting before (4A) and after (4B) exposure to leaking hydrogen gas according to embodiments of the present invention;

FIG. 3 shows a spectrographic response curve of the change in relative light transmittance of the tape product as it is exposed to hydrogen gas according to embodiments of the present invention;

FIG. 4a-c, are plan views illustrating different patterns of carbon thread dependent on the tensile strength and modulus of elasticity desired self-fusing tape with an embedded carbon fiber according to embodiments of the present invention.

### DETAILED DESCRIPTION OF THE INVENTION

It is to be understood that the figures and descriptions of the present invention have been simplified to illustrate elements that are relevant for a clear understanding, while eliminating, for the purpose of clarity, many other elements found in the self-fusing hydrogen indicating tape with or without an embedded carbon fiber technology, methods of using the same and its manufacture. Those of ordinary skill in the art may recognize that other elements and/or steps may be desirable in implementing the present invention. However, because such elements and steps are well known in the art, and because they do not facilitate a better understanding of the present invention, a discussion of such elements and steps is not provided herein.

In one non limiting embodiment the invention includes a self-sealing silicone tape product having one or more strips of silicone rubber tape and embedded in the silicone chemochromic metal oxide particles of tungsten oxide or molybdenum oxide, and a catalyst of metallic platinum or silver. Notably, the catalyst coating is applied to each microscopic metal oxide particle, as nano-scale metallic particles.

More particularly, the hydrogen gas indicating tape is made of a silicone polymer, which is highly permeable to hydrogen gas and hydrogen sulfide gas. Hydrogen gas or hydrogen sulfide gas from a leak can readily permeate into the silicone tape and react with the indicator pigment dispersed therein. The silicone resin used in the tape is a matrix which contains the dispersed chemically reactive indicating pigment powder and catalyst. The silicone matrix provides protection for the pigment from handling and from the weather, while still allowing the gases such as hydrogen and hydrogen sulfide to diffuse into the tape where the hazardous gas chemically reacts with the pigment to produce the telltale color change.

The pigment is a fine powder of a metal oxide to which a suitable catalyst is applied to facilitate the reaction between the metal oxide powder and the hazardous gas. By way of example and not limitation, the metal oxide is tungsten oxide or molybdenum oxide and the catalyst is metallic platinum or silver. The catalyst is a coating applied to each microscopic metal oxide particle as very fine, nano-scale metallic particles. The hazardous gas reacts with the metal oxide to chemically reduce it. Whereas the metal oxide itself is nearly colorless, the partially reduced oxide is highly colored blue or black.

In another embodiment the invention includes a method of making a self-sealing silicone tape that changes color in the presence of hydrogen or hydrogen sulfide, including the steps of: mixing a reinforced silicone polymer and nanoparticles of metal catalyst material with powder microparticles of chemochromic metal oxide material; and coating the powder microparticles of chemochromic material with the nanoparticles of the metal catalyst material by one of ginding or milling the nanoparticles of metal catalyst material with the microparticles of chemochromic metal oxide material.

In one embodiment of the invention the nanoparticles of the catalyst material are sized in a range of a 2 nanometers to 30 nanometers. In another embodiment of the invention the nanoparticles of the catalyst material are sized in a range of a 31 nanometers to a tens of nanometers. In yet another embodiment of the invention the microparticles of chemochromic metal oxide material are sized in a range of 10 to 200 microns in diameter.

With respect to other embodiments of a self-sealing silicone indicator tape used in the invention a catalyst material can be selected from the group of platinum, palladium, rhodium, nickel, combinations of these metals, or alloys of these materials with other metals.

More particularly, the silicone self-sealing tape incorporates a hydrogen sensor coating, including powder particles of chemochromic, transition metal oxide with a catalyst to create chemochromic powder pigments for use in paints, inks, dyes, and other emulsions that can be spread on surfaces to function as hydrogen detectors. A further embodiment of the present invention may include a chemochromic powder pigment silicone self-sealing tape that incorporates a powder particles of a transition metal oxide and a catalyst that is coated on or attached to surfaces of the transition metal oxide powder particles. Each of the foregoing hydrogen sensing coatings are as described in the related U.S. Pat. No. 9,422,160, entitled Visual Hydrogen Sensors Using Chemochromic Metal Oxide Microparticles Superficially Coated With Catalyst.

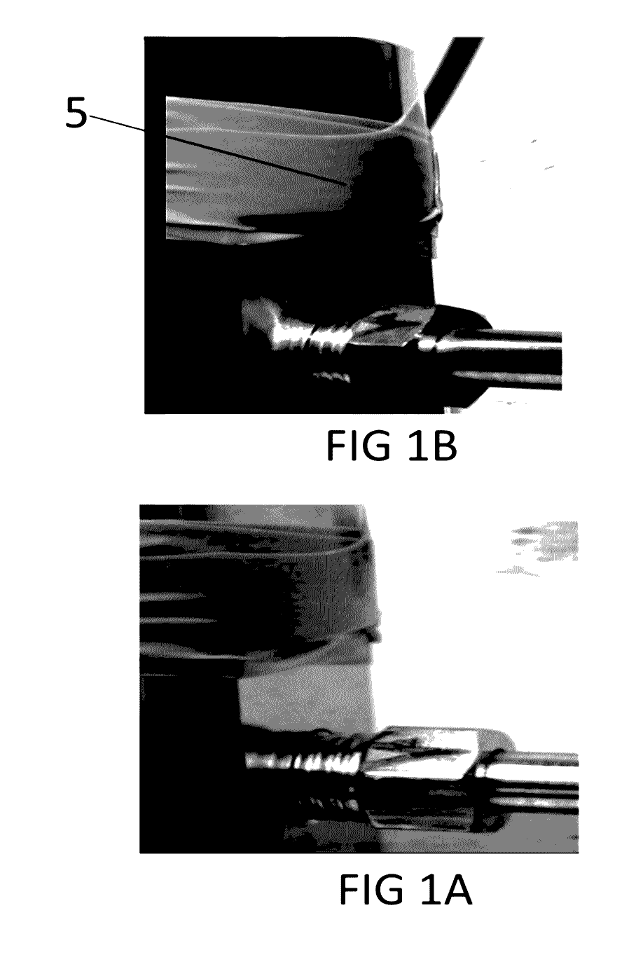

FIG. 1A is an illustration of a self-sealing silicone indicator tape used by way of example and not limitation in a high pressure hydrogen facility, know as a Hydrogen Fueling Station Gas Management Panel. This type of equipment is shipped and thereafter utilized in hydrogen fueling stations to calibrate hydrogen delivery pumps. As the equipment is moved during shipment, it undergoes vibration and stresses which can loosen fittings and cause leaks. Wrapping the potential leak sites with the indicator tape provides a quick and simple way to visually check for leaks at the new destination.

By way of example and not limitation FIG. 1A shows the tape as initially applied. FIG. 1B shows the tape after the system has been pressurized with hydrogen and a dark spot has formed on the tape indicating a hydrogen leak. The test illustrated in FIG. 1A and FIG. 1B is one of a number of ongoing tests of the hydrogen indicating tape being conducted at the US Department of Energy's National Renewable Energy Laboratory (NREL) in their Safety Sensor Testing Laboratory (see, Passive Leak Detection Using Commercial Hydrogen Colorimetric Indicator, Kevin Hartmann, et al, prepared under Task No. HT12.7210, National Renewable Energy Laboratory NREL, U.S. Department of Energy Office of Energy Efficiency & Renewable Energy. Report available from the National Renewable Energy Laboratory at www.nrel.gov/publications. September 2016.)

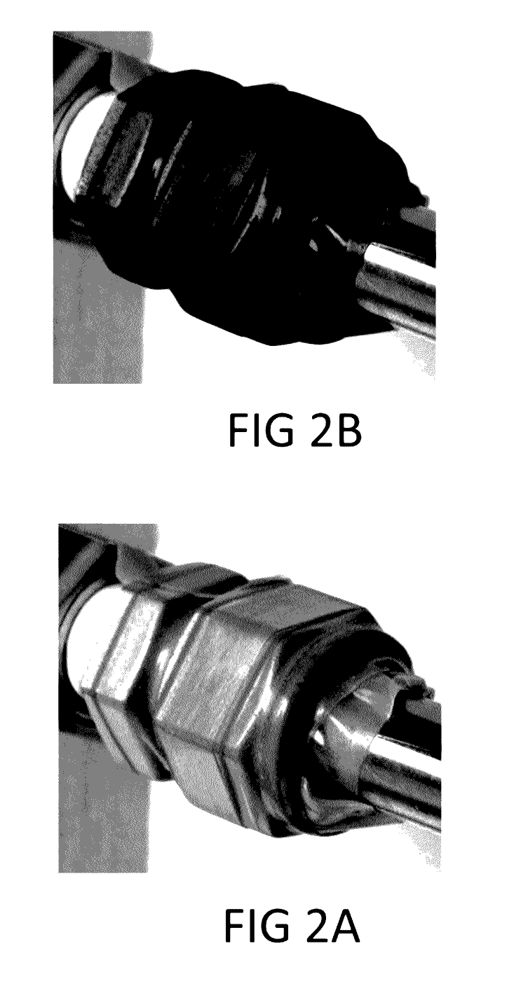

By way of example and not limitation, FIG. 2A and FIG. 2B show the self-sealing silicone indicator tape applied to a fitting before and after exposure to a hydrogen leak, respectively.

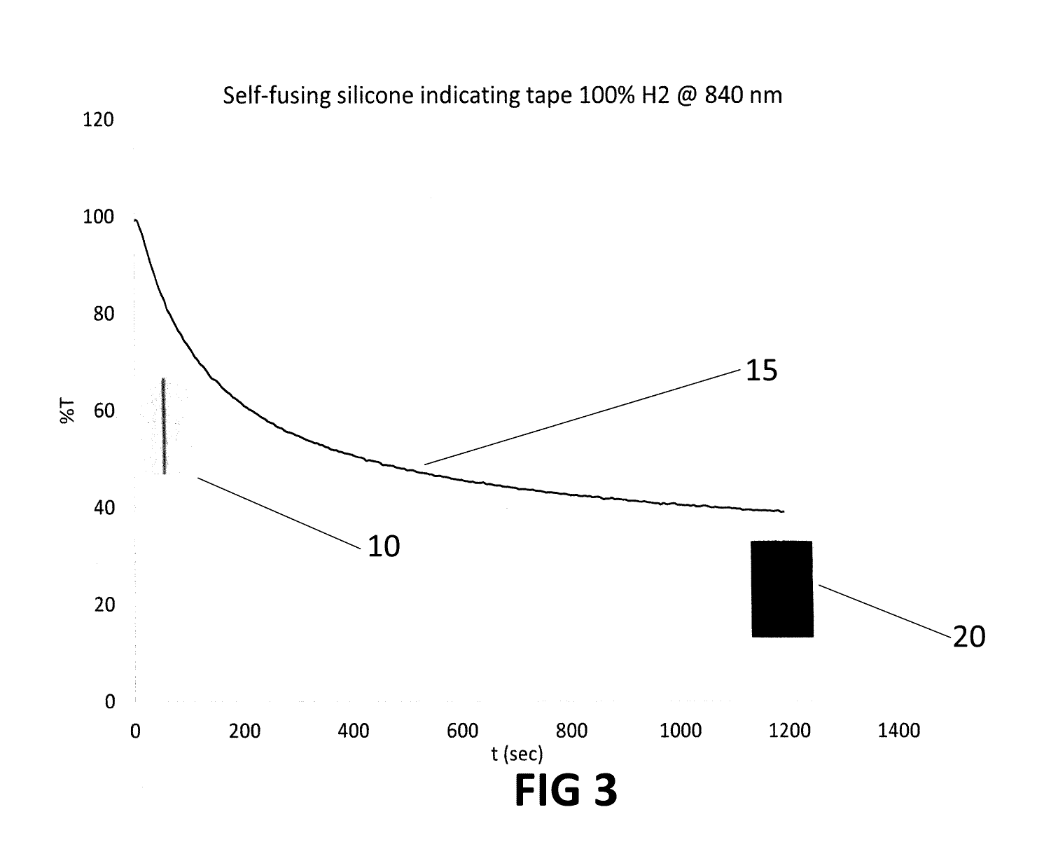

FIG. 3 shows a spectrographic response curve 15 of the change in tape relative light transmittance “T” along the vertical axis, as it is exposed to hydrogen gas according to embodiments of the present invention. Also shown is the corresponding visual color change 10 before and change 20 after exposure to hydrogen. Experiments have determined that a decrease of as small as 10% to 20% in the relative light transmittance is readily observable to the naked eye.

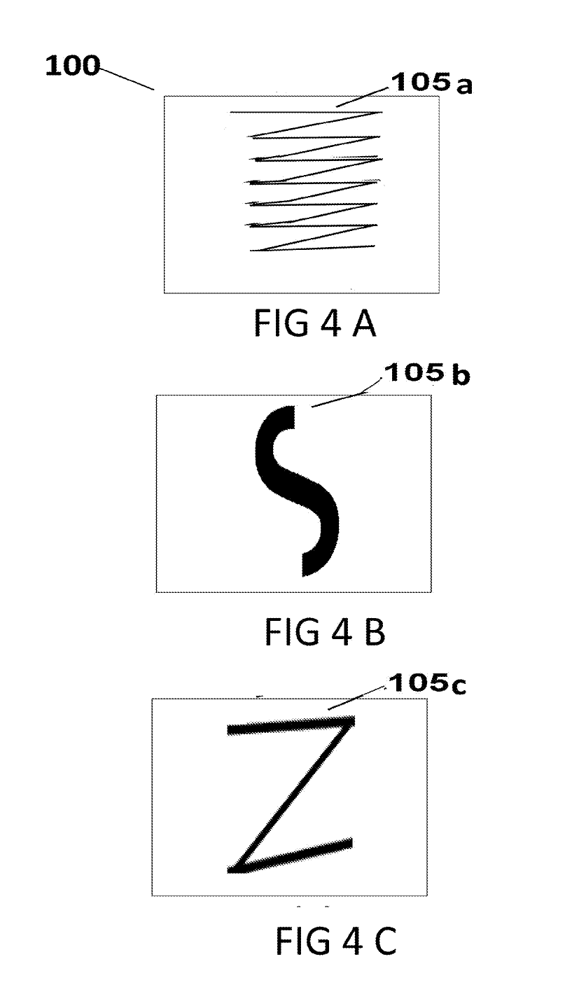

In FIG. 4a-c, a carbon fiber thread as illustrated as reference 105a, 105b, 105c, in multiple strands may be incorporated in the silicone self-fusing indicator tape 100 to provide added lengthwise tensile strength. The fiber-reinforcement tape may be particularly hard to tear or cut to the needed length. To facilitate tearing the tape into useful length, the tape may be perforated across its width at periodic distances along its length. Of course, if the tape is reinforced with carbon thread, the perforations will have to cut through all of the carbon fibers.

As further illustrated in FIG. 4a-c, the tape 100 incorporates two opposing elongated strips of silicone rubber and interposes therein the carbon fiber, such as embodied in thread 105a, which has a transverse spatial pattern such that the thread increases the tensile strength and increases the tear resistance, while maintaining the elastic limit of the strips of self-fusing tape. The incorporation of the carbon fiber in itself increases the tear resistance of the product, since the carbon fiber thread is virtually impossible to break.

The embodiments shown as “S”, triangular or a “Z” pattern in FIG. 4a-c, are by way of example and not limitation, cross sectional views illustrate different pattern of carbon thread 105a, 105b and 105c, dependent on the desired tensile strength and modulus of elasticity.

In certain industrial applications, a coloration may be added to the tape as a whole or as a marking along the length of the tape to provide additional information to the user and/or to increase its visibility in the field. The tape may be color coded with additional pigment or dye to indicate which hazardous gas it is intended to detect. Fluorescent pigment or dye may be added to the tape to enhance its visibility, as by way of example in the dark.

A self-fusing silicone suitable for the top layer of silicone tape and the bottom layer of silicone tape should endeavor to meet the requirements for operating at a continuous temperature between −64° C. to +260° C.; having a tensile strength of 700 psi minimum (ASTM-d-412 standard testing), thickness tolerances +/−, 002″ width +/−0.0625″; ultimate elongation 300% minimum (ASTM d-412 standard testing); and a tear resistance 85 psi.

An apparatus for manufacturing self-sealing silicone tape having a carbon fiber includes: a multilayered silicone tape that changes color in the presence of hydrogen or hydrogen sulfide, that changes color in the presence of hydrogen or hydrogen sulfide, a bobbin for the insertion of a spool having carbon thread for feeding a continuous strand of thread into an oscillating mechanism for moving the thread strand transverse to the motion of a top layer of silicone tape and a bottom layer of silicone tape that serve to encapsulate the carbon fiber thread for producing a desired spatial pattern that increases the tear resistance of the tape product, while insuring the elastic limit of the strips of the tape is maintained, each such spool of silicon tape supplied by bobbins, whereby when the carbon thread is embedded between the layers of silicone tape and a heat mechanism to supply a contact heat required to fuse the interior surfaces of the layer of silicone tape thereby encapsulating the carbon fiber thread. Methods of constructing and operating spools of silicon tape supplied by bobbins in the manner described are well known to those skilled in the art of manufacturing tape products.

A method of manufacturing silicone tape product that serves as an indicator tape that changes color in the presence of hydrogen or hydrogen sulfide, and that encapsulates the carbon fiber thread includes, providing an apparatus to embed a carbon thread between the two elongated strips of self-fusing silicone rubber tape, oscillating carbon thread as it enters a nib in a pattern that increases the tear resistance while insuring the elastic limit of the strips tape is maintained, heating the two elongated strips until the surfaces are permanently joined to encapsulate the carbon thread.

While the present invention has been described with reference to the illustrative embodiments, this description is not intended to be construed in a limiting sense. Various modifications of the illustrative embodiments, as well as other embodiments of the invention, will be apparent to those skilled in the art in reference to this description. It is expressly intended that all combinations of those elements that perform substantially the same function in substantially the same way to achieve the same results are within the scope of the invention.

We claim:

1. A self-sealing silicone tape product comprising: one or more strips of silicone rubber tape, having embedded in the silicone a chemochromic metal oxide particles of tungsten oxide or molybdenum oxide, and a one of a catalyst of metallic platinum or silver, wherein the catalyst coating applied to each microscopic metal oxide particle as nano-scale metallic particles.

2. The product of claim 1, wherein the nanoparticles of the catalyst material are sized in a range of 2 nanometers to 30 nanometers.

3. The product of claim 1, wherein the nanoparticles of the catalyst material are sized in a range of 31 nanometers to tens of nanometers.

4. The product of claim 1, wherein the microparticles of chemochromic metal oxide material are sized in a range of 10 to 200 microns in diameter.

5. The product of claim 1, wherein one of a silicone tape layer has embedded therein a carbon fiber thread having a transverse spatial pattern such that the thread increases the tear resistance while insuring the elastic limit of the strips of tape is maintained.

6. The product of claim 5, wherein the transverse spatial pattern is in the form of triangular steps.

7. The product of claim 5, wherein the transverse spatial pattern is in an “S” shape.

8. The product of claim 5, wherein the transverse spatial pattern is in the form of rectangular steps.

9. The product of claim 5, wherein the transverse spatial pattern is in a “Z” shape.

10. The product of claim 1, wherein the silicone rubber has a tensile strength exceeding 700 psi.

11. The product of claim 1, wherein the silicone rubber has an ultimate elongation of 300% minimum.

12. The product of claim 1, wherein periodic perforations across the width of the tape may be added to facilitate the tearing or cutting of convenient lengths of the tape.

13. The product of claim 1, wherein a coloration is added to the tape to provide one of additional information to a user or to increase the tape visibility in the field.

14. The product of claim 1, wherein a color code having one of an additional pigment or dye indicates which hazardous gas the product is intended to detect.

15. The product of claim 1, wherein one of a fluorescent pigment or dye enhances visibility.

16. A method of making a self-sealing silicone tape that changes color in the presence of hydrogen or hydrogen sulfide, comprising: mixing a reinforced silicone polymer and nanoparticles of a metal catalyst material with powder microparticles of chemochromic metal oxide material; and coating the powder microparticles of chemochromic material with the nanoparticles of the metal catalyst material by one of ginding or milling the nanoparticles of metal catalyst material with the microparticles of chemochromic metal oxide material.

17. The method of claim 16, wherein the microparticles of chemochromic metal oxide material are less than 100 microns in diameter.

18. The method of claim 16, wherein the nanoparticles of the catalyst material are sized in a range of a 16 nanometers to 30 nanometers.

19. The method of claim 16, wherein the nanoparticles of the catalyst material are sized in a range of 31 nanometers to tens of nanometers.

20. The method of claim 16, wherein the microparticles of chemochromic metal oxide material are sized in a range of 10 to 200 microns in diameter.

21. The method of claim 16, wherein a silicone tape layer has embedded therein a carbon fiber thread having a transverse spatial pattern such that the thread increases the tear resistance while insuring the elastic limit of the strips of tape is maintained.

22. The method of claim 21, wherein the transverse spatial pattern is in the form of triangular steps.

23. The method of claim 21, wherein the transverse spatial pattern is in an “S” shape.

24. The method of claim 21, wherein the transverse spatial pattern is in the form of rectangular steps.

25. The method of claim 21, wherein the transverse spatial pattern is in a “Z” shape.

26. The method of claim 21, wherein the silicone rubber has a tensile strength exceeding 700 psi.

27. The method of claim 21, wherein the silicone rubber has an ultimate elongation of 300% minimum.

28. An apparatus for manufacturing self-sealing silicone tape having a carbon fiber includes: a multilayered silicone tape that changes color in the presence of hydrogen or hydrogen sulfide, a bobbin for the insertion of a spool having carbon thread for feeding a continuous strand of thread into an oscillating mechanism for moving the thread strand transverse to the motion of a top layer of the silicone tape and a bottom layer of silicone tape that serve to encapsulate the carbon fiber thread for producing a desired spatial pattern that increases the tear resistance of the tape, while insuring the elastic limit of the strips of the tape is maintained, each such spool of silicone tape supplied by bobbins, whereby when the carbon thread is embedded between the layers of silicone tape and a heat mechanism to supply a contact heat required to fuse the interior surfaces of the layer of silicone tape thereby encapsulating the carbon fiber thread.
